(19) United States
(12) Patent Application Publication
(10) Pub. No.: US 2010/0114287 A1
(43) Pub. Date: May 6, 2010

(54) IMPLANTABLE TISSUE MARKER ELECTRODE

(76) Inventors: Salvatore Privitera, Mason, OH (US); James R. Edgerton, Plano, TX (US); David J. Drachman, West Chester, OH (US); R. Scott Robinson, Cincinnati, OH (US); Kenneth Lance Miller, Hamilton, OH (US)

Correspondence Address: COOK ALEX LTD, SUITE 2850, 200 WEST ADAMS STREET, CHICAGO, IL, 60606, US

(21) Appl. No.: 12/609,219

(22) Filed: Oct. 30, 2009

Related U.S. Application Data
(60) Provisional application No. 61/109,552, filed on Oct. 30, 2008.

(51) Int. Cl. A61N 1/05; A61B 5/042

(52) U.S. Cl. 607/130; 607/129; 600/375

(57) ABSTRACT

The present application discloses a tissue marker that may be permanently applied to cardiac (or other) tissue by means such as, but not limited to, a minimally-invasive procedure to allow for pre- and post-op lesion site testing, with the marker also preferably being radiopaque to facilitate post-op imaging. More specifically, the marker may preferably comprise or include an electrode as part of an integrated assembly. The marker may be mounted on the tissue with a suitable tissue retention member for securing the marker in place. The disclosed examples include one or more tissue retention members, and in an exemplary embodiment comprises a pair of clips for securing the assembly to a target tissue. Each retention member or clip has a conductive lead with an electrically conductive surface in the form of a patch associated therewith. Each clip is preferably associated with a discrete electrically conductive area on the surface of the patch so that the assembly may function as a bi-polar electrode, with a voltage applied between the discrete conductive areas.

### CROSS-REFERENCE TO RELATED APPLICATION

This application claims the benefit of the filing date of U.S. Provisional Application Ser. No. 61/109,552, filed Oct. 30, 2008, the entire contents of which are incorporated by reference.

### BACKGROUND

Methods and apparatus have been developed for treating atrial fibrillation by creating lines of scar tissue that pose an interruption in the path of errant electrical impulses in the heart tissue. Scar tissue may be created by, e.g., surgical cutting of the tissue, freezing of the tissue by cryogenic probe, heating the tissue via RF energy, and other technologies. Methods and apparatus for creating transmural lines of ablation using RF energy are shown and described in, e.g., U.S. Pat. No. 6,517,536, U.S. Pat. No. 6,974,454, and U.S. Pat. No. 7,393,353, which are incorporated herein by reference.

Various methods for determining the efficacy of the lines of ablation have been developed using pacing and sensing electrodes. See U.S. Pat. No. 6,905,498, also incorporated herein by reference. For example, if a pacing pulse or signal is applied to cardiac tissue on one side of a line of ablation, but not sufficiently detected by an EKG sensor located on the tissue on the other side of the line of ablation, the line of ablation may be deemed effective for blocking electrical impulses. There may also be instances in which it is desirable to post-surgically locate the line of ablation for further testing its efficacy at blocking electrical impulses.

### SUMMARY

The present application discloses a tissue marker that may be permanently applied to cardiac (or other) tissue by means such as, but not limited to, a minimally-invasive procedure to allow for pre- and post-op lesion site testing, with the marker also preferably being radiopaque to facilitate post-op imaging. More specifically, the marker may preferably comprise or include an electrode as part of an integrated assembly. The marker may be mounted on the tissue with a suitable tissue retention member for securing the marker in place. The disclosed examples include one or more tissue retention members, and in an exemplary embodiment comprises a pair of clips for securing the assembly to a target tissue. Each retention member or clip has a conductive lead with an electrically conductive surface in the form of a patch associated therewith. Each clip is preferably associated with a discrete electrically conductive area on the surface of the patch so that the assembly may function as a bi-polar electrode, with a voltage applied between the discrete conductive areas.

An applicator is also disclosed that comprises a hand piece, an elongated shaft sized to be passed through a trocar, and a delivery mechanism in the form of a spring-loaded push rod for dispensing the clips of the marker.

A housing is also disclosed that is adapted to be received on the distal end of the shaft of the applicator, and may be pre-loaded with the marker.

### BRIEF DESCRIPTION OF DRAWINGS

FIG. 1 is a perspective view of a marker in accordance with one embodiment in the present disclosure.

FIG. 2 is a perspective view of a first embodiment of a clip suitable for use in the marker/electrode assembly described herein.

FIG. 3 is a plan view of the clip of FIG. 2.

FIG. 4 is an end view of the clip of FIG. 2.

FIG. 5 is a side view of the clip of FIG. 2.

FIG. 6 is a plan view of a second embodiment of a clip suitable for use in the marker assembly described herein.

FIG. 7 is a side view of the clip of FIG. 6.

FIG. 8 is a plan view of a patch electrode forming part of the marker.

FIG. 9 is a plan view of the patch material for forming the patch electrode prior to being incorporated into the marker.

FIG. 10 is a further pre-assembly view of the patch electrode material.

FIG. 11 is a perspective view showing an applicator for use in deploying a marker that is described in the present disclosure.

FIG. 12 is an exploded perspective view of the distal end of the applicator assembly showing the details of the push rod, in conjunction with the clip housing and two clips, the patch and leads forming the marker.

FIG. 13 is a perspective view of the push rod forming part of the applicator.

FIG. 14 is an end view of the push rod of FIG. 15.

FIG. 15 is a perspective view of the housing for use in conjunction with the applicator assembly and marker.

FIG. 16 is a top or proximal end view of the housing of FIG. 17.

FIG. 17 is a bottom or distal end view of the housing of FIG. 17.

FIG. 18 is a cross-sectional view of the housing showing the camming surfaces on the interior thereof for engaging the legs of the clip members.

### DESCRIPTION

In keeping with the disclosure, a marker for application to tissue is provided that comprises a patch with an electrically conductive surface for contacting the target tissue. At least one retainer is associated with the patch for securing the electrically conductive surface in contact with the target tissue. A conductive lead is in conductive contact with the conductive surface of the patch for transmitting and receiving electrical impulses

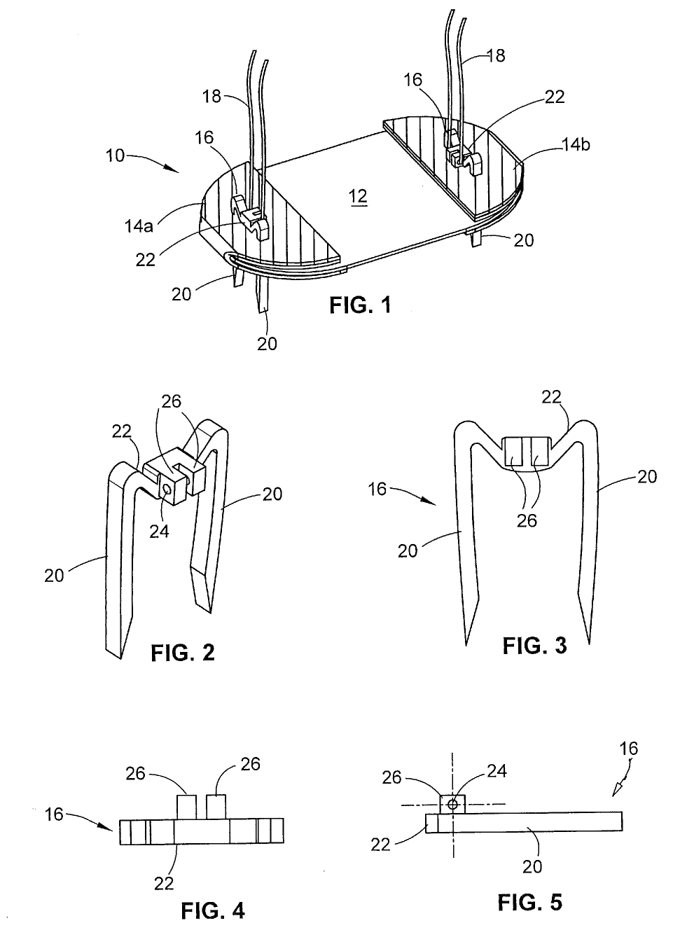

Turning to the figures of the drawings, there is seen in FIG. 1 a perspective view of one embodiment of a marker 10 in accordance with the present disclosure. The marker 10 has an electrically conductive surface adapted to contact the target tissue in the form of a patch 12. Preferably, the patch 12 has two discrete electrically conductive areas or surfaces 14a, 14b that are insulated from one another so that a voltage can be applied between them, thus permitting the patch 12 to serve as a bi-polar electrode. However, the patch 12 may have a single, continuous electrically-conductive surface if a mono-polar electrode is desired.

The electrically conductive surfaces 14a, 14b may be a metal/metal chloride film that is applied to the patch 12 by, e.g., printing or other suitable way such as soldering, adhesive or using techniques known in the manufacture of microprocessors, such as lithography and the like. Alternatively, the electrode surface may be formed by interweaving into the patch 12 strands of an electrically-conductive, biocompatible material, such as stainless steel. As shown in FIG. 1, the tissue contacting areas 14a, 14b are on both the upper and lower surfaces of the patch 12. To provide a tissue contacting surface sufficiently large for conducting pacing and sensing impulses, each electrically-conductive surface 14a, 14b on the patch preferably has a tissue-contacting surface area of at least about 2 mm², but smaller or larger areas may be used depending on the particular arrangement of the conductive areas or members.

In the illustrated embodiment, a clip member 16 is associated with each conductive area 14a, 14b of the patch 12 for securing the patch 12 to the target tissue. A conductive contact or lead 18 is also associated with each of the electrically conductive surface 14a, 14b of the patch 12 to transmit electrical impulses to and from the patch 12. The lead 18 may comprise stainless steel, copper, gold, silver, or other conductive materials. The lead is preferably braided and is insulated except for the portion containing the clip and/or patch. If the patch 12 has a single continuous electrically conductive surface, only a single clip 12 and lead 18 are required.

With reference to FIGS. 2-5, a first embodiment of a clip 16 for use in the marker of the present disclosure is seen. The clip 16 is generally U-shaped with a pair of legs 20 depending from a base or bridging segment 22. When the target tissue for the marker 10 is the heart, the legs 20 of the clip 16 are sized in length so as to not completely penetrate through the myocardium. For such an application, the clip 16 is, preferably, on the order of approximately 5 mm in overall length, with the legs 20 of the clip being on the order of approximately 3.5 mm in length and the bridge/base 22 being on the order of approximately 3.3 mm in length. The legs 20 of the clip may optionally include a barb (not shown) to more securely fix the clip 16 to the target tissue.

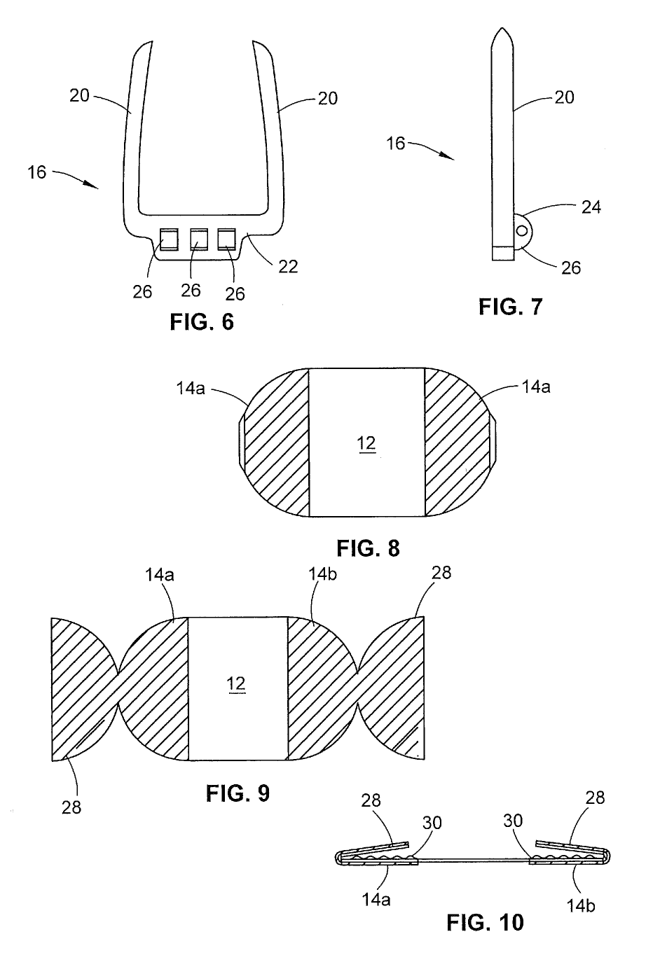

The base/bridge portion 22 of the clip 16 is configured to receive the electrically conductive lead 18. This may take any suitable form, such as apertures 24 through which the conductive lead 18 is threaded. In the embodiment of FIGS. 2-5, the base member includes a pair of brackets 26, each having an aperture 24 therein through which the lead 18 may be threaded. Alternatively, as shown in FIGS. 6 and 7, the base/bridging portion 22 of the clip 16 may be formed with a series of three apertures 24. In either case, the conductive lead 18 is preferably threaded through apertures 24 so that both ends of the lead 18 extend out of the surgical field for attachment to a pacing/sensing generator. As a further option, the bridge portion 22 of the clip 16 may be formed without any apertures, and the lead 18 may be simply threaded between the bridge portion 22 of the clip 16 and the upper surface of the patch 12. If, at the end of a procedure the leads 18 need to be removed, this may be simply done by pulling on one end of the lead 18.

Suitable material for the clips 16 include any biocompatible implantable material that exhibits the requisite closure force to prevent inadvertent dislodgment, such as titanium, stainless steel, plated copper, platinum, or any other conductive biocompatible material. The clip materials are also preferably radiopaque, so that, if the marker 10 is permanently implanted, the clip 16 may serve to enhance visibility of the clip through fluoroscopy.

As illustrated, the clips 16/leads 18 are associated with the patch electrode 12 through which the legs 20 of the clips 16 extend. The patch 16 serves to optimize the electrode surface area and epicardial contact. Further, the patch 12 provides for immediate wound closure and minimizes the risk of the marker 10 migrating or dislodging once deployed. The patch 12 also facilitates long-term tissue regeneration.

With reference to FIGS. 8-10, the illustrated patch 12 electrode is generally oval in shape and is made of a material such as knit braid polyester. A mesh material is preferred for the patch 12 in order to promote tissue ingrowth. The electrically conductive coating 14a, 14b, is applied to two discrete areas of the patch 12 electrically isolated from the other, each area having a clip 16 and lead 18 associated therewith. The electrode surfaces 14a, 14b are preferably spaced apart approximately 2 mm. The overall length of the patch 12 is preferably no more than about 5 mm and the width is preferably no more than approximately 3 mm.

Both the top and bottom surfaces of the patch 12 are preferably coated with the electrically conductive material to ensure good conduct between the leads 18 and the electrode surface 14a, 14b. To this end, the patch 12 may be formed as shown in FIG. 9 with ends 28 that fold back onto the back side of the patch 12 so as to present two discrete conductive surfaces for engagement with the leads 18. As shown in FIG. 10, the ends 28 may be secured to the back side of the patch by an adhesive 30, which is preferably an electrically conductive pressure sensitive adhesive. The adhesive 30 is applied to the upper surface of the patch by, e.g., printing, and then the ends 28 of the patch 12 are folded back onto the top side (as shown in FIG. 10).

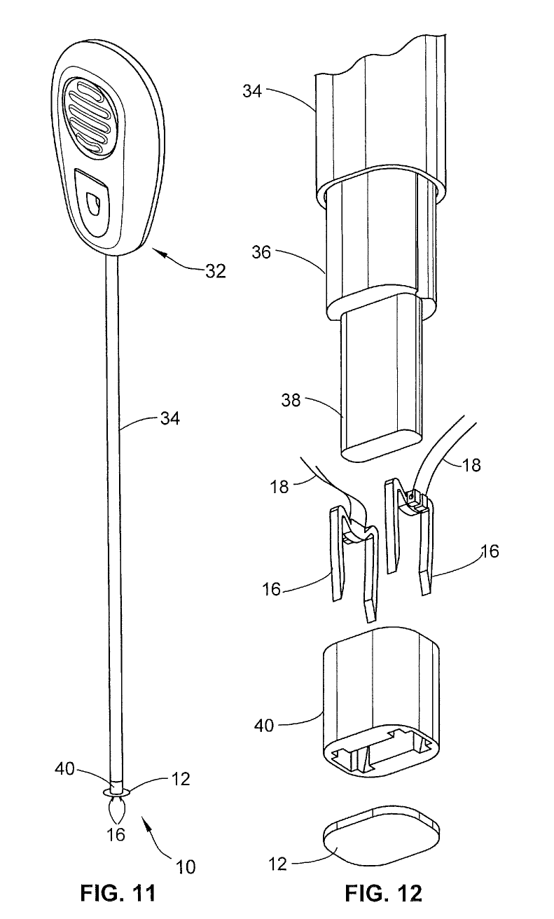

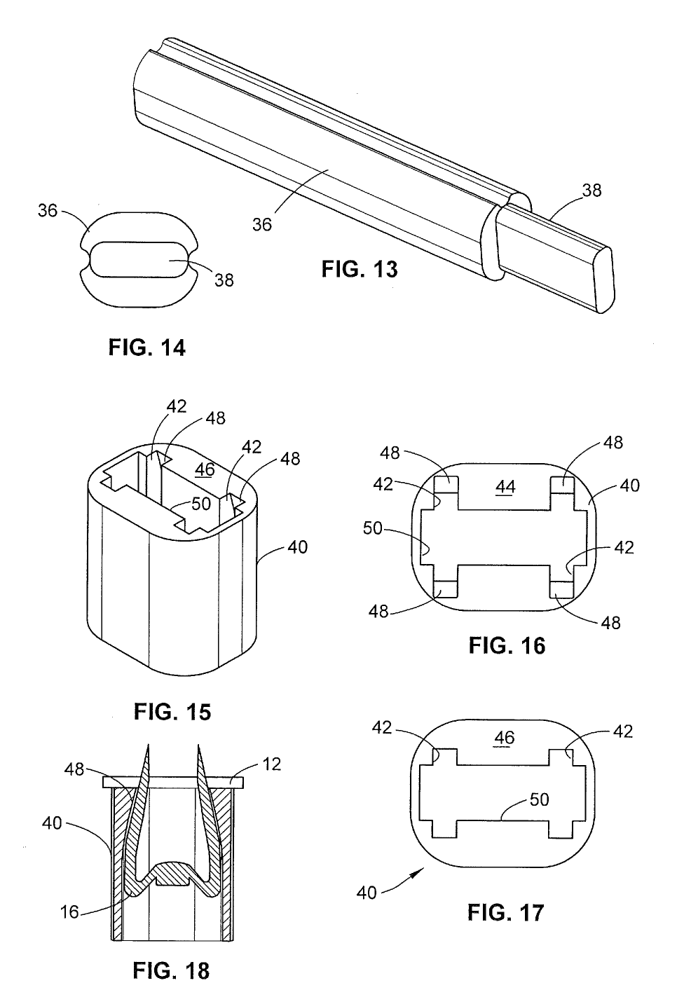

An applicator 32 for applying markers 10 in accordance with the present disclosure is seen in FIGS. 11-18. The tool 32 includes an elongated shaft 34 that is preferably sized to fit through a 5 mm trocar and slidably mounts a push rod 36 therein (best seen in FIG. 12-14). A marker 10 is associated with the distal end of the applicator 32. The distal end of the push rod 36 includes a driver segment 38 that is received on the interior of a clip housing 40 upon actuation of the applicator 32 to eject the clips 16 from the housing 40 and secure the marker 10 to the target tissue.

One or more markers 10 as described above are pre-loaded onto the housing 40. With reference to FIGS. 15-18, the housing 40 receives the proximal portion of the clips 16 in spaced-apart parallel slots 42. The slots 42 narrow in width from the proximal side 44 of the housing 40 to the distal side 46 of the housing 40, thus providing a camming surface 48 (best seen in FIG. 18). The camming surface 48 acts to close the legs 20 of the clips 16 toward each other when the clip is ejected from the housing 40, thus securing the marker to the target tissue. The clip housing 40 has a further slot 50 transverse to the slots 42 into which the driving segment 38 of the push rod 36 is slidably received. As can be appreciated, the driving segment 38 of the push rod 36 engages the bridge portions 22 of the clips 16. When axially extended, the push rod 36 pushes the clips out of the housing 90. Preferably, when a marker is loaded into the housing 40, the legs 20 of the clip extend at least partially out of the housing 40 and the leads are accessible such that the marker is fully functional. Thus, electrical readings may be taken before the device is deployed to assist in the optimally locating the marker 10 on the target tissue before it is affixed thereto.

In order to apply a marker 10 according to the present disclosure to the surface of a heart or other target tissue, the distal end of the applicator 32 is brought into contact with the target tissue in proximity to the line of ablation or lesion, and then the applicator 32 is activated to discharge the clips and secure the marker 10 to the target tissue. A single marker 10 may be used with the two conductive surfaces of the patch being on opposite sides of the line of ablation. Alternatively, two markers may be applied to the target tissue, with one being on each side of the line of ablation. As noted above, if ends of the legs 20 of the clips 16 extend out from the housing 40 so that they are able to contact the target tissue prior to attachment, readings may be taken or pulses delivered to confirm the positioning of the marker 10 prior to being fixed to the target tissue. Although disclosed for contacting the epicardial surface, the marker may also be used in contact with the endocardial surface, although such may require a different delivery system.

Once a marker has been fixed with one electrically conductive surface 14a, 14b on each side of a line of ablation (or, e.g., a monopolar marker is secured on each side of a line of ablation), the efficacy of the line of ablation can be determined by using one of the conductive surfaces to transmit pacing pulses to the cardiac tissue, and the other conductive surface as an EKG sensor. Pacing signals are then transmitted to the cardiac tissue through the pacing electrode. If the pacing signals are detected by the EKG electrode, then the line of ablation is not complete or transmural. Conversely, if the pacing pulses are not sufficiently detected by the EKG electrode, the line of ablation may be deemed transmural.

Having shown and described various examples of an embodiment according to the present disclosure, further adaptations of the methods, components and systems described herein may be accomplished by appropriate modifications by one of ordinary skill in the art without departing from the scope of the disclosure. Several of such potential modifications have been mentioned, and still others will be apparent to those skilled in the art. For instance, the examples, embodiments, geometries, materials, dimensions, steps, and the like discussed above are illustrative and are not necessarily required. Accordingly, the scope of the present disclosure should be considered in terms of the following claims and is understood not to be limited to the details of structure and operation shown and described in the specification and drawings.

1. A marker for application to a target tissue comprising:
a patch having an electrically-conductive surface for contacting the target tissue;
at least one retainer associated with the patch for securing the electrically-conductive surface in contact with the target tissue; and
a conductive lead in conductive contact with the electrically conductive surface of the patch for transmitting and receiving electrical impulses.

2. A marker in accordance with claim 1 in which a portion of the marker is radiopaque.

3. The marker of claim 1 in which the conductive lead is secured to the marker by the retainer.

4. The marker of claim 1 in which the retainer comprises a clip having a pair of depending legs for extending through the patch and penetrating into the target tissue.

5. The marker of claim 1 in which the electrically conductive surface comprises two discrete electrically conductive surfaces with a retainer and conductive lead associated with each electrically conductive surfaces.

6. The marker of claim 5 in which the conductive leads are secured to the marker by their associated retainers.

7. The marker of claim 5 in which the retainers comprise clips, each clip having a pair of depending legs for extending through the patch and penetrating into the target tissue.

8. The marker of claim 3 in which the retainer comprises an eyelet for receiving the conductive lead.

9. The marker of claim 6 in which the retainer comprises an eyelet for receiving the conductive lead.

10. The marker of claim 2 in which at least a portion of the patch is radiopaque.

11. The marker of claim 5 in which at least a portion of the patch is radiopaque.

12. The marker of claim 2 wherein at least a portion of the retainer is radiopaque.

13. The marker of claim 5 wherein at least a portion of the retainer is radiopaque.

14. A combination marker/electrode assembly for application to a target tissue comprising:
a patch having electrically-conductive surface thereon for contacting the target tissue;
at least one clip associated with the patch for securing the patch to the target tissue, the clip having a pair of depending legs extending through the patch for penetrating into the target tissue and being made of a radiopaque material.
a conductive lead associated with the clip and in conductive contact with the electrically conductive surface of the patch for transmitting and receiving electrical impulses.

15. The combination of claim 14 wherein the clip comprises an eyelet for receiving the conductive lead.

16. The continuation of claim 14 herein at least one of the patch and clip is radiopaque.
